(19) United States
(12) Patent Application Publication
(10) Pub. No.: US 2022/0117184 A1
(43) Pub. Date: Apr. 21, 2022

(54) Method for Producing Sterile Cannabis Sativa L. Cultivars

(71) Applicant: Green Point Research Holdings LLC, Fort Lauderdale, FL (US)

(72) Inventors: Will Wadlington, Fort Lauderdale, FL (US); Mike Dukes, Fort Lauderdale, FL (US); Jack Light Townsend, JR., Fort Lauderdale, FL (US)

(73) Assignee: Green Point Research Holdings LLC

(21) Appl. No.: 17/503,946

(22) Filed: Oct. 18, 2021

Related U.S. Application Data
(60) Provisional application No. 63/093,129, filed on Oct. 16, 2020.

(51) Int. Cl. A01H 6/28; A01H 3/04; A01H 5/12

(52) U.S. Cl. CPC A01H 6/28; A01H 5/12; A01H 3/04

(57) ABSTRACT

The Method of Producing Sterile Cannabis sativa L. Cultivars includes applying a novel colchicine treatment to axillary meristems of diploid cannabis plants to produce sterile cannabis growth. The colchicine may be a range of concentrations applied over a 24 hour period to produce the sterile cannabis growth. The sterile cannabis plant will not reproduce sexually but may be replicated via other methods that are used to replicate plants.

### CROSS-REFERENCE TO RELATED APPLICATION

This application claims the benefit of U.S. Provisional Patent App. No. 63/093,129 filed on Oct. 16, 2020, titled Method for Producing Sterile, Triploid Cannabis sativa L. Cultivars, the disclosure of which is hereby incorporated by reference.

### FIELD

The present invention generally relates to methods for developing polyploid and sterile C. sativa plants and the sterile C. sativa plant variety generated from the disclosed methods.

### BACKGROUND

Historically, C. sativa is one of the oldest domesticated crops, with evidence that is has been cultivated by several ancient civilizations. It has served as a multiuse plant, providing the world with a source for fibers, textiles, medical remedies, and recreational uses over the past 50 centuries. Most modern taxonomists consider C. sativa to be the only species in the genus Cannabis. Thus, the common name ‘cannabis’ sufficiently describes all forms of C. sativa, including both industrial hemp and marijuana. In the United States (and most western countries), the cannabis plant has a storied past—in 1938, the Marihuana Tax Act placed cannabis cultivation under the purview of the United States Drug Enforcement Administration. Federally legal cannabis, in the form of industrial hemp, has experienced a recent commercial resurgence due to its descheduling pursuant to the Agriculture Improvement Act of 2018.

Hemp and marijuana are typically separated by their respective tetrahydrocannabinol (“THC”) contents. Although a level of about 1% THC is considered the minimum amount to induce an intoxicating effect in humans, the threshold for classifying cannabis as hemp is 0.3% THC. Cannabis with a higher THC concentration is currently legally considered to be marijuana. While cannabis with a lower concentration of THC is named Cannabis Sativa L. or commonly referred to as “industrial hemp.” Throughout this disclosure, any mention of “cannabis” refers to the legal form of cannabis: C. sativa.

Cannabis is a dioecious annual plant, with staminate male flowers or pistillate female flowers on separate individuals. The plant is widely cultivated for unique pharmacological properties due to the presence of a vast range of high value, versatile molecules—cannabinoids and terpenes—which accumulate in glandular trichromes.

One problem in growing Cannabis sativa L. intended for cannabinoid production is uncontrolled pollination. The highest cannabinoid content is found on unpollinated, seedless female flowers. Unintended pollination from external pollen drift from other male flowers or females that can occasionally develop male flowers can result in yield loss. To guard against uncontrolled pollination, farmers and growers expend significant effort to prevent pollination in cannabis growing environments.

Accordingly, there is a need for a sterile cannabis that eliminates uncontrolled pollination and limits the depletion of cannabinoids due to seed production.

### SUMMARY

This disclosure comprises methods of creating polyploid cannabis plants with odd sets of chromosomes.

In one embodiment, a method of producing a sterile Cannabis sativa plant, the method comprises applying a treatment of a colchicine solution to an axillary meristem of a four-leaf cannabis plant having two levels of growth.

In some embodiments, the colchicine solution applied to the axillary meristem is a concentration between 0.1 percent by weight and 0.9 percent by weight.

In some embodiments, the method includes applying a second treatment of the colchicine solution to the axillary meristem of the four-leaf cannabis plant within 24 hours of the first treatment.

In some embodiments, the method includes applying a third treatment of the colchicine solution to the axillary meristem of the four-leaf cannabis plant within 24 hours of the first treatment.

In some embodiments, the axillary meristems are treated with a volume of the colchicine solution that is between 10 μL to 250 μL.

In some embodiments, the sterile cannabis plant produced by colchicine solution application is triploid.

In some embodiments, the method includes, after treatment with colchicine, cutting an apical meristem of the cannabis plant and placing the cannabis plant in a dark room for up to 48 hours.

In another exemplary embodiment, a method for producing sterile Cannabis sativa L. cultivars includes the steps of applying a first volume of a colchicine solution to an axillary meristem of a cannabis plant at least at a four-leaf growth stage; applying a second volume of the colchicine solution to the axillary meristem of a cannabis plant; and applying a third volume of the colchicine solution to the axillary meristem of a cannabis plant; wherein the first volume, the second volume, and the third volume are applied within a 24 hour period.

In another exemplary embodiment, a method of producing a sterile cannabis plant includes the steps of treating a first cannabis plant with a colchicine solution to produce a polyploid cannabis plant; providing a second cannabis plant; and mating the polyploid cannabis plant with the second cannabis plant to produce the sterile cannabis plant.

In another exemplary embodiment, the method of producing a sterile, triploid cannabis plant includes the steps of providing a cannabis plant having at least two levels of growth or in a four-leaf growth stage; applying three treatments of colchicine solution to an axillary meristem of the cannabis plant, the three treatments being applied in a single 24 hour span; and producing a new growth from the axillary meristem that is triploid, sterile cannabis.

### BRIEF DESCRIPTION OF THE DRAWINGS

FIG. 1 is an illustration of two cannabis plants at different growth stages.

### DETAILED DESCRIPTION

This disclosure presents methods to produce sterile cannabis plants. Previous methods rely on tissue culture or treatment of germinating seeds and plants which had only developed to the 2-leaf stage (code 1002) to produce polyploid cannabis. The disclosed methods generate polyploids from plants that have reached at least the 4-leaf stage (code 1004). (The ‘codes’ are a system used to describe growth phases of hemp plants. See Mediavilla, Vito, Manuel Jonquera, Ingrid Schmid-Slembrouck and Alberto Soldati 1998. A decimal code for growth stages of hemp (Cannabis sativa L.). Journal of the International Hemp Association 5(2): 65, 68-74.).

In various embodiments discussed herein, sterile varieties of C. sativa were developed from seedlings, clones, and reciprocal crossing of an artificially created polyploid with a wildtype diploid.

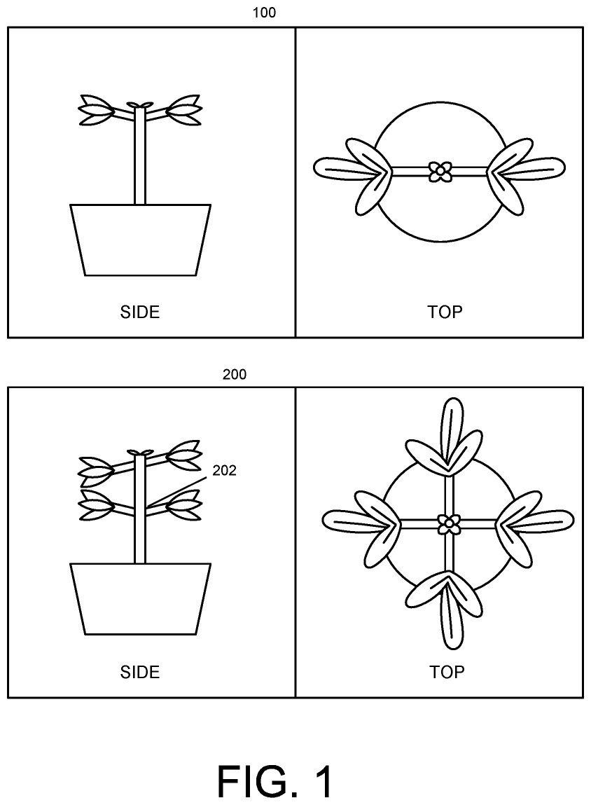

Referring to FIG. 1, a plant at the 2-leaf stage 100 and a plant at the 4-leaf stage 200 are shown. In one exemplary embodiment of the Method of Producing Sterile Cannabis sativa L. Cultivars, an axillary meristem 202 of a wildtype, diploid cannabis plant 200 is treated with colchicine to produce polyploid growth which is then allowed to grow before removing the known diploid growth from the plant. In some embodiments, the wildtype, diploid cannabis plant is rooted with two levels of growth. In some embodiments, the cannabis plant 200 has grown to at least the 4-leaf stage. The cannabis plant to be treated may be rooted and have reached at least the 4-leaf stage of growth. By way of example, a cannabis plant with a second level of growth, with two leaves at each level, and sufficient rooting may be treated with a colchicine solution.

In one exemplary embodiment, when an axillary meristem 202 of the cannabis plant 200 at the 4-leaf stage is treated with colchicine, triploid growth is induced. Then, the triploid growth is allowed to grow before removing any remaining diploid growth. Diploid growth refers to any leaves or shoots of diploid growth prior to treatment of the axillary meristem 202 with colchicine.

In some embodiments, polyploid plants are created by treating the axillary meristems 202 of 1-month old seedlings (code 1004, or 4-leaves on two levels of growth) with a colchicine solution that is in the range of 0.1-1.0% by weight.

In some embodiments, the colchicine solution may be applied to the axillary meristem 202 three times in 24 hours. The colchicine solution may be applied at a volume from 10 μL to 250 μL at each of the three treatment times.

By way of example, a 4-leaf cannabis seedling may be treated with 50 μL of a 0.2% by weight colchicine solution, applied to the axillary meristems 202, three times within 24 hours, to create a triploid cannabis plant.

In some embodiments, the treated seedlings may be pruned to have apical meristems removed then placed in a dark room for 48 hours to encourage lateral growth immediately after treatment. After dark treatment, plants may then be moved to a greenhouse with 45% shade and with supplemental 16-hour lighting to maintain vegetative phase. After 2 weeks, new growth from the treated axillary buds will have polyploid chromosomes.

In other embodiments of the Method, triploids were created by crossing artificially produced polyploid cannabis plants with diploid genotypes. For example, a polyploid cannabis plant produced through treatment of colchicine may be induced to flower and cross or mate with a diploid cannabis plant. In some embodiments, a female diploid cannabis plant may be sex-reversed to become a pollen producer and mated with a female polyploid cannabis plant, created by treatment with colchicine, to produce a sterile female cannabis plant. In some embodiments, a female polyploid cannabis plant, created by treatment with colchicine, may be sex-reversed to become pollen donator and mated with a female diploid cannabis plant to produce a sterile female cannabis plant. In some embodiments, the sterile female cannabis plant produced through this method are triploid.

In some embodiments, the sterile female cannabis plant produced through the disclosed methods will produce sterile seeds. The sterile seeds may then be planted and grow into a sterile female cannabis plant. The sterile seeds develop and harden and can be planted without embryo rescue.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A method of producing a sterile Cannabis sativa plant, the method comprising:
applying a treatment of a colchicine solution to an axillary meristem of a four-leaf cannabis plant.

2. The method of claim 1, wherein the colchicine solution is between 0.1 percent by weight and 0.9 percent by weight.

3. The method of claim 1, further comprising:
applying a second treatment of the colchicine solution to the axillary meristem of the four-leaf cannabis plant within 24 hours of the first treatment.

4. The method of claim 3, further comprising:
applying a third treatment of the colchicine solution to the axillary meristem of the four-leaf cannabis plant within 24 hours of the first treatment.

5. The method of claim 1, wherein the colchicine solution is 0.2 percent by weight.

6. The method of claim 5, further comprising:
the treatment includes applying a volume of 10 μL to 250 μL of the colchicine solution.

7. The method of claim 1, further comprising 50 μL of the colchicine solution is applied at the treatment.

8. The method of claim 1, further comprising after treatment with colchicine:
producing a sterile cannabis plant.

9. The method of claim 8, wherein the sterile cannabis plant is triploid.

10. The method of claim 1, further comprising after treatment with colchicine:
cutting an apical meristem of the cannabis plant; and
placing the cannabis plant in a dark room for up to 48 hours.

11. A method for producing sterile Cannabis sativa, the method comprising:
applying a first volume of a colchicine solution to an axillary meristem of a cannabis plant at least at a four-leaf growth stage;
applying a second volume of the colchicine solution to the axillary meristem of a cannabis plant; and
applying a third volume of the colchicine solution to the axillary meristem of a cannabis plant;
wherein the first volume, the second volume, and the third volume are applied within a 24 hour period.

13. The method of claim 12, wherein the colchicine solution is between 0.1 and 0.9 percent by weight.

14. The method of claim 12, wherein the colchicine solution is 0.2 percent by weight.

15. The method of claim 12, wherein the first volume, the second volume, and the third volume are between 10 μL to 250 μL.

16. The method of claim 14, wherein the first volume, the second volume, and the third volume are 50 μL.

17. The method of claim 12, further comprising after applying the third volume:
cutting an apical meristem of the cannabis plant; and
placing the cannabis plant in a dark room for up to 48 hours.

18. A method of producing a sterile cannabis plant, the method comprising:
treating a first cannabis plant with a colchicine solution to produce a polyploid cannabis plant;
providing a second cannabis plant; and
mating the polyploid cannabis plant with the second cannabis plant to produce the sterile cannabis plant.

19. The method of claim 18, wherein the sterile cannabis plant is a triploid.
